(19) United States
(12) Patent Application Publication
(10) Pub. No.: US 2016/0319319 A1
(43) Pub. Date: Nov. 3, 2016

(54) METHOD FOR THE RECOMBINANT PRODUCTION OF A POLYPEPTIDE IN PROKARYOTIC CELLS

(71) Applicant: Hoffmann-La Roche Inc., Little Falls, NJ (US)

(72) Inventor: CHRISTIAN SCHANTZ, PENZBERG (DE)

(73) Assignee: Hoffmann-La Roche Inc., Little Falls, NJ (US)

(21) Appl. No.: 15/009,588

(22) Filed: Jan. 28, 2016

Related U.S. Application Data
(63) Continuation of application No. PCT/EP2014/066261, filed on Jul. 29, 2014.

(30) Foreign Application Priority Data
Jul. 31, 2013 (EP) 13178739.2

(51) Int. Cl. C12P 21/00; C12N 15/70; C12N 9/02

(52) U.S. Cl. CPC C12P 21/00; C12N 9/0036; C12Y 106/99003; C12N 15/70

(57) ABSTRACT

Herein is reported a method for the recombinant production of a polypeptide in E. coli comprising the steps of i) cultivating an NADH dehydrogenase II-deficient E. coli expressing the polypeptide, and ii) recovering the polypeptide from the cell or the cultivation medium.

### CROSS REFERENCE TO RELATED APPLICATIONS

This application is a continuation of International Application No. PCT/EP2014/066261 having an international filing date of Jul. 29, 2014, the entire contents of which are incorporated herein by reference, and which claims benefit under 35 U.S.C. §119 to European Patent Application No. 13178739.2 filed Jul. 31, 2013.

### SEQUENCE LISTING

The instant application contains a Sequence Listing submitted via EFS-Web and hereby incorporated by reference in its entirety. Said ASCII copy, created on Jan. 27, 2016, is named P31703USSeqList.txt, and is 2,920 bytes in size.

### FIELD OF THE INVENTION

Herein is reported a prokaryotic cell genetically modified by knockout of the NADH dehydrogenase II gene (ndh-gene) and its use in the production of a polypeptide.

### BACKGROUND OF THE INVENTION

In recent years the production of proteins has steadily increased and it is likely that proteins will become the biggest group of therapeutics available for the treatment of various diseases in the near future. The impact of proteins emerges from their specificity, such as the specific target recognition and binding function.

Cell cultures are used in fermentative processes to produce substances, in particular proteins. A distinction is made between processes in which the cell cultures are genetically unmodified and form their own metabolic products and processes in which the organisms are genetically modified in such a manner that they either produce a larger amount of their own substances such as proteins or produce foreign (heterologous) substances. The organisms producing the substances are supplied with a nutrient medium which guarantees the survival of the organisms and enables the production of the desired target compound. Numerous culture media are known for these purposes which enable an optimal cultivation of the specific host.

High-cell-density cultivation of Escherichia coli is reported by Riesenberg (Riesenberg, D., et al., Curr. Opin. Biotechnol. 2 (1991) 380-384) and Horn (Horn, U., et al., Appl. Microbiol. Biotechnol. 46 (1996) 524-532). Riesenberg, D. and Guthke, R. (Appl. Microbiol. Biotechnol. 51 (1999) 422-430) reported the high-cell-density cultivation of microorganisms. Growing E. coli to high cell density is reviewed by Shiloach, J. and Fass, R. (Biotechnol. Advances 23 (2005) 345-357).

The energetic efficiency of Escherichia coli-effects of mutations in components of the aerobic respiratory chain is reported by Calhoun et al. (J. Bacteriol. 175 (1993) 3020-3025). Melo et al. (Microbiol. Mol. Biol. Rev. 68 (2004) 603-616) report new insights into type II NAD(P)H:quinone oxidoreductases. Enhancement of lactate and succinate formation in adhE or pta-ackA mutants of NADH dehydrogenase-deficient Escherichia coli is reported by Yun et al. (J. Appl. Microbiol. 99 (2005) 1404-1412).

Design, construction and performance of the most efficient biomass producing E. coli bacterium is reported by Trinh et al. (Met. Eng. 8 (2006) 628).

### SUMMARY OF THE INVENTION

It has been found that by the deletion/inactivation of the ndh-gene, which codes for the enzyme NADH dehydrogenase II, a genetically modified prokaryotic organism can be obtained that has, when compared to the parent strain that is isogenic except for the ndh-gene, comparable oxygen uptake rates, comparable growth rates but has an increased productivity. Thus, it has been found that by the deletion/inactivation of the ndh-gene the specific productivity of a prokaryotic organism can be increased.

One aspect as reported herein is a method for the recombinant production of a polypeptide in a prokaryotic cell comprising the following steps:
cultivating a prokaryotic cell expressing the polypeptide (i.e. cultivating a cell that comprises a nucleic acid encoding the polypeptide), and
recovering the polypeptide from the cell or the cultivation medium, wherein the prokaryotic cell is deficient in the ndh-gene (i.e. the prokaryotic cell comprises/contains no functional copy of the ndh-gene).

One aspect as reported herein is a method for the recombinant production of a polypeptide in a prokaryotic cell comprising the following steps:
cultivating a prokaryotic cell expressing the polypeptide (i.e. cultivating a cell that comprises a nucleic acid encoding the polypeptide), and
recovering the polypeptide from the cell or the cultivation medium, wherein the prokaryotic cell is deficient in the ndh-gene (i.e. the prokaryotic cell comprises/contains no functional copy of the ndh-gene), and wherein the polypeptide is not an enzyme.

One aspect as reported herein is a method for the recombinant production of a polypeptide in a prokaryotic cell comprising the following steps:
cultivating a prokaryotic cell expressing the polypeptide (i.e. cultivating a cell that comprises a nucleic acid encoding the polypeptide), and
recovering the polypeptide from the cell or the cultivation medium,
   wherein the prokaryotic cell is deficient in the ndh-gene (i.e. the prokaryotic cell comprises/contains no functional copy of the ndh-gene),
   and wherein the polypeptide is not a respiratory chain pathway enzyme or a polypeptide encoded by an antibiotic resistance inducing gene.

One aspect as reported herein is a method for the recombinant production of a polypeptide in a prokaryotic cell comprising the following steps:
cultivating a prokaryotic cell expressing the polypeptide (i.e. cultivating a cell that comprises a nucleic acid encoding the polypeptide), and
recovering the polypeptide from the cell or the cultivation medium,
   wherein the prokaryotic cell is deficient in the ndh-gene (i.e. the prokaryotic cell comprises/contains no functional copy of the ndh-gene),
   and wherein the polypeptide is not a NADH dehydrogenase, a SoxM type oxidase, a Sox type oxidase, a cytochrome bd type oxidase, a cytochrome bo type oxidase or any polypeptide encoded by an antibiotic resistance inducing gene.

One aspect as reported herein is a method for the recombinant production of a polypeptide in a prokaryotic cell comprising the following steps:
cultivating a prokaryotic cell expressing the polypeptide (i.e. cultivating a cell that comprises a nucleic acid encoding the polypeptide), and
recovering the polypeptide from the cell or the cultivation medium,
   wherein the prokaryotic cell is deficient in the ndh-gene (i.e. the prokaryotic cell comprises/contains no functional copy of the ndh-gene),
   and wherein the prokaryotic cell has the genotype thi-1, Δndh, ΔpyrF, acnA, aceA, icd, wherein the acnA gene encoded polypeptide comprises a S68G mutation, the aceA gene encoded polypeptide comprises a S522G mutation and the icd gene encoded polypeptide comprises a D398E and a D410E mutation.

One aspect as reported herein is a method for the recombinant production of a polypeptide in a prokaryotic cell comprising the following steps:
cultivating a prokaryotic cell expressing the polypeptide (i.e. cultivating a cell that comprises a nucleic acid encoding the polypeptide), and
recovering the polypeptide from the cell or the cultivation medium,
   wherein the prokaryotic cell is deficient in the ndh-gene (i.e. the prokaryotic cell comprises/contains no functional copy of the ndh-gene),
   and wherein the polypeptide is an immunoglobulin, an immunoglobulin fragment, an immunoglobulin-toxin conjugate, an immunoglobulin fragment-toxin conjugate, a toxin, a cytokine or a hormone.

One aspect as reported herein is a method for the production of a polypeptide in a prokaryotic cell comprising the following steps:
cultivating a prokaryotic cell expressing the polypeptide (i.e. cultivating a cell that comprises a nucleic acid encoding the polypeptide), and
recovering the polypeptide from the cell or the cultivation medium,
   wherein the prokaryotic cell is deficient in the ndh-gene (i.e. the prokaryotic cell comprises/contains no functional copy of the ndh-gene),
   and wherein the polypeptide is not an enzyme.

One aspect as reported herein is a method for the production of a polypeptide in a prokaryotic cell comprising the following steps:
cultivating a prokaryotic cell expressing the polypeptide (i.e. cultivating a cell that comprises a nucleic acid encoding the polypeptide), and
recovering the polypeptide from the cell or the cultivation medium,
   wherein the prokaryotic cell is deficient in the ndh-gene (i.e. the prokaryotic cell comprises/contains no functional copy of the ndh-gene),
   and wherein the polypeptide is not a respiratory chain pathway enzyme or a polypeptide encoded by an antibiotic resistance inducing gene.

One aspect as reported herein is a method for the production of a polypeptide in a prokaryotic cell comprising the following steps:
cultivating a prokaryotic cell expressing the polypeptide (i.e. cultivating a cell that comprises a nucleic acid encoding the polypeptide), and
recovering the polypeptide from the cell or the cultivation medium,
   wherein the prokaryotic cell is deficient in the ndh-gene (i.e. the prokaryotic cell comprises/contains no functional copy of the ndh-gene),
   and wherein the polypeptide is not a NADH dehydrogenase, a SoxM type oxidase, a Sox type oxidase, a cytochrome bd type oxidase, a cytochrome bo type oxidase or any polypeptide encoded by an antibiotic resistance inducing gene.

One aspect as reported herein is a method for the production of a polypeptide in a prokaryotic cell comprising the following steps:
cultivating a prokaryotic cell expressing the polypeptide (i.e. cultivating a cell that comprises a nucleic acid encoding the polypeptide), and
recovering the polypeptide from the cell or the cultivation medium,
   wherein the prokaryotic cell is deficient in the ndh-gene (i.e. the prokaryotic cell comprises/contains no functional copy of the ndh-gene),
   and wherein the prokaryotic cell has the genotype thi-1, Δndh, ΔpyrF, acnA, aceA, icd, wherein the acnA gene encoded polypeptide comprises a S68G mutation, the aceA gene encoded polypeptide comprises a S522G mutation and the icd gene encoded polypeptide comprises a D398E and a D410E mutation.

One aspect as reported herein is a method for the production of a polypeptide in a prokaryotic cell comprising the following steps:
cultivating a prokaryotic cell expressing the polypeptide (i.e. cultivating a cell that comprises a nucleic acid encoding the polypeptide), and
recovering the polypeptide from the cell or the cultivation medium,
   wherein the prokaryotic cell is deficient in the ndh-gene (i.e. the prokaryotic cell comprises/contains no functional copy of the ndh-gene),
   and wherein the polypeptide is an immunoglobulin, an immunoglobulin fragment, an immunoglobulin-toxin conjugate, an immunoglobulin fragment-toxin conjugate, a toxin, a cytokine or a hormone.

The product of the ndh-gene is the NADH dehydrogenase II.

In one embodiment the prokaryotic cell is further deficient in the bd-type oxidase.

In one embodiment the prokaryotic cell is an E. coli cell. In one embodiment the E. coli is an E. coli K12.

In one embodiment the method is a high cell density cultivation.

In one embodiment the prokaryotic cell that is deficient in the ndh-gene (NADH dehydrogenase II) as a comparable oxygen uptake rate (OUR) when compared to a prokaryotic cell that has the same genotype except that it has a functional ndh-gene (NADH dehydrogenase II). That is, the only genetic difference between the ndh-deficient cell and reference cell is the ndh-deficiency.

In one embodiment the prokaryotic cell that is deficient in the ndh-gene (NADH dehydrogenase II) has a comparable growth rate compared to a prokaryotic cell that has the same genotype except that it has a functional ndh-gene (NADH dehydrogenase II).

In one embodiment the prokaryotic cell that is deficient in the ndh-gene (NADH dehydrogenase II) as a higher production rate when compared to a prokaryotic cell that has the same genotype except that it has a functional ndh-gene (NADH dehydrogenase II). In one embodiment the production rate is the specific production rate.

In one embodiment the method comprises after the cultivation step the following steps:
incubating the cultivation medium including the cells at a temperature of 40° C. or higher for at least 10 minutes, and
recovering the insoluble polypeptide from the cells and/or the cultivation medium and thereby producing the polypeptide.

In one embodiment the incubating is at a temperature between 40° C. and 60° C.

In one embodiment the incubating is at a temperature of 45° C. or higher. In one embodiment the incubating is at a temperature of about 45° C.

In one embodiment the incubating is for 10 minutes to 180 minutes.

One aspect as reported herein is a method for the recombinant production of a polypeptide in E. coli comprising the following steps:
cultivating an NADH dehydrogenase II-deficient E. coli expressing the polypeptide (i.e. cultivating an NADH dehydrogenase II-deficient E. coli comprising a nucleic acid encoding the polypeptide), and
recovering the polypeptide from the cell or the cultivation medium.

One aspect as reported herein is a method for the recombinant production of a polypeptide in E. coli comprising the following steps:
cultivating an NADH dehydrogenase II-deficient E. coli expressing the polypeptide (i.e. cultivating an NADH dehydrogenase II-deficient E. coli comprising a nucleic acid encoding the polypeptide), and
recovering the polypeptide from the cell or the cultivation medium,
   and wherein the polypeptide is not an enzyme.

One aspect as reported herein is a method for the recombinant production of a polypeptide in E. coli comprising the following steps:
cultivating an NADH dehydrogenase II-deficient E. coli expressing the polypeptide (i.e. cultivating an NADH dehydrogenase II-deficient E. coli comprising a nucleic acid encoding the polypeptide), and
recovering the polypeptide from the cell or the cultivation medium,
   and wherein the polypeptide is not a respiratory chain pathway enzyme or a polypeptide encoded by an antibiotic resistance inducing gene.

One aspect as reported herein is a method for the recombinant production of a polypeptide in E. coli comprising the following steps:
cultivating an NADH dehydrogenase II-deficient E. coli expressing the polypeptide (i.e. cultivating an NADH dehydrogenase II-deficient E. coli comprising a nucleic acid encoding the polypeptide), and
recovering the polypeptide from the cell or the cultivation medium,
   and wherein the polypeptide is not a NADH dehydrogenase, a SoxM type oxidase, a Sox type oxidase, a cytochrome bd type oxidase, a cytochrome bo type oxidase or any polypeptide encoded by an antibiotic resistance inducing gene.

One aspect as reported herein is a method for the recombinant production of a polypeptide in E. coli comprising the following steps:
cultivating an NADH dehydrogenase II-deficient E. coli expressing the polypeptide (i.e. cultivating an NADH dehydrogenase II-deficient E. coli comprising a nucleic acid encoding the polypeptide), and
recovering the polypeptide from the cell or the cultivation medium,
   and wherein the NADH dehydrogenase II-deficient E. coli has the genotype thi-1, Δndh, ΔpyrF, acnA, aceA, icd, wherein the acnA gene encoded polypeptide comprises a S68G mutation, the aceA gene encoded polypeptide comprises a S522G mutation and the icd gene encoded polypeptide comprises a D398E and a D410E mutation.

One aspect as reported herein is a method for the recombinant production of a polypeptide in E. coli comprising the following steps:
cultivating an NADH dehydrogenase II-deficient E. coli expressing the polypeptide (i.e. cultivating an NADH dehydrogenase II-deficient E. coli comprising a nucleic acid encoding the polypeptide), and
recovering the polypeptide from the cell or the cultivation medium,
   and wherein the polypeptide is an immunoglobulin, an immunoglobulin fragment, an immunoglobulin-toxin conjugate, an immunoglobulin fragment-toxin conjugate, a toxin, a cytokine or a hormone.

One aspect as reported herein is a method for the production of a polypeptide in E. coli comprising the following steps:
cultivating an NADH dehydrogenase II-deficient E. coli expressing the polypeptide (i.e. cultivating an NADH dehydrogenase II-deficient E. coli comprising a nucleic acid encoding the polypeptide), and
recovering the polypeptide from the cell or the cultivation medium.

One aspect as reported herein is a method for the production of a polypeptide in E. coli comprising the following steps:
cultivating an NADH dehydrogenase II-deficient E. coli expressing the polypeptide (i.e. cultivating an NADH dehydrogenase II-deficient E. coli comprising a nucleic acid encoding the polypeptide), and
recovering the polypeptide from the cell or the cultivation medium,
   and wherein the polypeptide is not an enzyme.

One aspect as reported herein is a method for the production of a polypeptide in E. coli comprising the following steps:
cultivating an NADH dehydrogenase II-deficient E. coli expressing the polypeptide (i.e. cultivating an NADH dehydrogenase II-deficient E. coli comprising a nucleic acid encoding the polypeptide), and
recovering the polypeptide from the cell or the cultivation medium,
   and wherein the polypeptide is not a respiratory chain pathway enzyme or a polypeptide encoded by an antibiotic resistance inducing gene.

One aspect as reported herein is a method for the production of a polypeptide in E. coli comprising the following steps:
cultivating an NADH dehydrogenase II-deficient E. coli expressing the polypeptide (i.e. cultivating an NADH dehydrogenase II-deficient E. coli comprising a nucleic acid encoding the polypeptide), and
recovering the polypeptide from the cell or the cultivation medium,
   and wherein the polypeptide is not a NADH dehydrogenase, a SoxM type oxidase, a Sox type oxidase, a cytochrome bd type oxidase, a cytochrome bo type oxidase or any polypeptide encoded by an antibiotic resistance inducing gene.

One aspect as reported herein is a method for the production of a polypeptide in E. coli comprising the following steps:
cultivating an NADH dehydrogenase II-deficient E. coli expressing the polypeptide (i.e. cultivating an NADH dehydrogenase II-deficient E. coli comprising a nucleic acid encoding the polypeptide), and
recovering the polypeptide from the cell or the cultivation medium,
   and wherein the NADH dehydrogenase II-deficient E. coli has the genotype thi-1, Δndh, ΔpyrF, acnA, aceA, icd, wherein the acnA gene encoded polypeptide comprises a S68G mutation, the aceA gene encoded polypeptide comprises a S522G mutation and the icd gene encoded polypeptide comprises a D398E and a D410E mutation.

One aspect as reported herein is a method for the recombinant production of a polypeptide in E. coli comprising the following steps:
cultivating an NADH dehydrogenase II-deficient E. coli expressing the polypeptide (i.e. cultivating an NADH dehydrogenase II-deficient E. coli comprising a nucleic acid encoding the polypeptide), and
recovering the polypeptide from the cell or the cultivation medium,
   and wherein the polypeptide is an immunoglobulin, an immunoglobulin fragment, an immunoglobulin-toxin conjugate, an immunoglobulin fragment-toxin conjugate, a toxin, a cytokine or a hormone.

In one embodiment the NADH dehydrogenase II-deficient E. coli has a comparable oxygen uptake rate as an E. coli with the same genotype except that it has a functional NADH dehydrogenase II.

In one embodiment the NADH dehydrogenase II-deficient E. coli has a comparable growth rate as an E. coli with the same genotype except that it has a functional NADH dehydrogenase II.

In one embodiment the NADH dehydrogenase II-deficient E. coli has a higher production rate as an E. coli with the same genotype except that it has a functional NADH dehydrogenase II.

In one embodiment the NADH dehydrogenase II-deficient E. coli is further deficient in the bd-type oxidase.

In one embodiment the NADH dehydrogenase II-deficient E. coli is an E. coli K12.

In one embodiment the NADH dehydrogenase II-deficient E. coli has the genotype thi-1, Δndh, ΔpyrF.

In one embodiment the NADH dehydrogenase II-deficient E. coli has the genotype thi-1, Δndh, ΔpyrF, acnA, aceA, icd.

In one embodiment the NADH dehydrogenase II-deficient E. coli has the genotype thi-1, Δndh, ΔpyrF, acnA, aceA, icd, wherein the acnA gene encoded polypeptide comprises a S68G mutation, the aceA gene encoded polypeptide comprises a S522G mutation and the icd gene encoded polypeptide comprises a D398E and a D410E mutation.

In one embodiment the NADH dehydrogenase II-deficient E. coli has a functional Zwf-gene.

In one embodiment the NADH dehydrogenase II-deficient E. coli has a functional ldhA-gene.

In one embodiment the NADH dehydrogenase II-deficient E. coli has a functional maeA-gene.

In one embodiment the NADH dehydrogenase II-deficient E. coli has a functional maeB-gene.

In one embodiment the NADH dehydrogenase II-deficient E. coli has a functional Zwf-gene, a functional ldhA-gene, a functional maeA-gene and a functional maeB-gene.

In one embodiment the method comprises after the cultivation step the following steps:
incubating the cultivation medium including the cells at a temperature of 40° C. or higher for at least 10 minutes, and
recovering the insoluble polypeptide from the cells and/or the cultivation medium and thereby producing the polypeptide.

In one embodiment the incubating is at a temperature between 40° C. and 60° C.

In one embodiment the incubating is at a temperature of 45° C. or higher. In one embodiment the incubating is at a temperature of about 45° C.

In one embodiment the incubating is for 10 minutes to 180 minutes.

One aspect as reported herein is an E. coli K12 that has the genotype thi-1, ΔpyrF Δndh.

One aspect as reported herein is an E. coli K12 that has the genotype thi-1, Δndh, ΔpyrF, acnA, aceA, icd, wherein the acnA gene encoded polypeptide comprises a S68G mutation, the aceA gene encoded polypeptide comprises a S522G mutation and the icd gene encoded polypeptide comprises a D398E and a D410E mutation.

One aspect as reported herein is the use of an NADH dehydrogenase II-deficient E. coli in the production of a recombinant polypeptide.

One aspect as reported herein is the use of an NADH dehydrogenase II-deficient E. coli in the production of a polypeptide.

In one embodiment the NADH dehydrogenase II-deficient E. coli has the genotype Δndh, thi-1, ΔpyrF. In one embodiment the NADH dehydrogenase II-deficient E. coli has the genotype thi-1, Δndh, ΔpyrF, acnA, aceA, icd.

In one embodiment the NADH dehydrogenase II-deficient E. coli has the genotype thi-1, Δndh, ΔpyrF, acnA, aceA, icd, wherein the acnA gene encoded polypeptide comprises a S68G mutation, the aceA gene encoded polypeptide comprises a S522G mutation and the icd gene encoded polypeptide comprises a D398E and a D410E mutation.

In one embodiment the NADH dehydrogenase II-deficient E. coli is further deficient in the bd-type oxidase.

### DETAILED DESCRIPTION OF THE INVENTION

Herein is reported a method for the (recombinant) production of a polypeptide using a prokaryotic cell that is deficient in the ndh-gene whereby due to the deficiency in the ndh-gene i) the oxygen uptake rate and the growth rate is comparable to the parent prokaryotic cell that is isogenic except for the deficiency in the ndh-gene, and ii) the specific production rate is increased compared to the parent prokaryotic cell that is isogenic except for the deficiency in the ndh-gene.

In one embodiment the prokaryotic cell is an Escherichia cell, or a Bacillus cell, or a Lactobacillus cell, or a Corynebacterium cell, or a Yeast cell (Saccharomyces, Candida, or Pichia). In a further embodiment the cell is an Escherichia coli cell, or a Bacillus subtilis cell, or a Lactobacillus acidophilus cell, or a Corynebacterium glutamicum cell, or a Pichia pastoris yeast cell.

In one embodiment the prokaryotic cell is an E. coli K12 cell or an E. coli B cell.

In one embodiment the prokaryotic cell is an E. coli K12 cell having the genotype: thi-1, ΔompT, ΔpyrF, acnA, aceA, icd (parental strain) and the genotype: thi-1, ΔompT, ΔpyrF, Δndh, acnA, aceA, icd (modified strain), wherein the acnA gene encoded polypeptide comprises a S68G mutation, the aceA gene encoded polypeptide comprises a S522G mutation and the icd gene encoded polypeptide comprises a D398E and a D410E mutation. In addition the parental and the modified strain lack the following e14 prophage genes: ymfD, ymfE, lit, intE, xisE, ymfI, ymfJ, cohE, croE, ymfL, ymfM, owe, ymfR, bee, jayE, ymfQ, stfP, tfaP, tfaE, stfE, pinE, mcrA.

Methods for cultivating a prokaryotic cell are known to a person of skill in the art (see e.g. Riesenberg, D., et al., Curr. Opin. Biotechnol. 2 (1991) 380-384). The cultivating can be with any method. In one embodiment the cultivating is a batch cultivating, a fed-batch cultivating, a perfusion cultivating, a semi-continuous cultivating, or a cultivating with full or partial cell retention.

In one embodiment the cultivating is a high cell density cultivating. The term “high cell density cultivating” denotes a cultivating method wherein the dry cell weight of the cultivated prokaryotic cell is at one point in the cultivating at least 10 g/L. In one embodiment the dry cell weight is at one point in the cultivating at least 20 g/L, or at least 50 g/L, or at least 100 g/L, or more than 100 g/L. In order to reach such a high cell density state the volume of feed and/or adjustment solutions added during the cultivating has to be as small as possible. Methods for the determination of dry cell weight are reported e.g. in Riesenberg, D., et al., Appl. Microbiol. Biotechnol. 34 (1990) 77-82.

The term “parent cell” denotes a cell, which has the same genotype as the deficient cell but the gene deficient in the deficient cell is functional in the parent cell. Thus, a parent cell and a deficient cell are isogenic except for the gene that is deficient.

The term “functional ndh-gene” denotes that the ndh-gene is transcribed and translated and the gene product, i.e. the NADH dehydrogenase II, is functional and enzymatic active.

The produced polypeptide can be any biologically active polypeptide.

The term “biologically active polypeptide” denotes an organic molecule, e.g. a biological macromolecule such as a peptide, protein, glycoprotein, nucleoprotein, mucoprotein, lipoprotein, synthetic polypeptide or protein, that causes a biological effect when administered in or to artificial biological systems, such as bioassays using cell lines and viruses, or in vivo to an animal, including but not limited to birds or mammals, including humans. This biological effect can be but is not limited to enzyme inhibition or activation, binding to a receptor or a ligand, either at the binding site or circumferential, signal triggering or signal modulation. Biologically active molecules are without limitation for example immunoglobulins, or hormones, or cytokines, or growth factors, or receptor ligands, or agonists or antagonists, or cytotoxic agents, or antiviral agents, or imaging agents, or enzyme inhibitors, enzyme activators or enzyme activity modulators such as allosteric substances. In one embodiment the polypeptide is an immunoglobulin, immunoglobulin conjugate, or an immunoglobulin fragment.

A “polypeptide” is a polymer consisting of amino acids joined by peptide bonds, whether produced naturally or synthetically. A polypeptide as defined herein consists of ten or more amino acids. A polypeptide may also comprise non-naturally occurring amino acid residues and/or non-amino acid components, such as carbohydrate groups, metal ions, or carboxylic acid esters. The non-amino acid components may be added by the cell, in which the polypeptide is expressed, and may vary with the type of cell.

Polypeptides are defined in terms of their amino acid backbone structure or the nucleic acid encoding the same. Additions such as carbohydrate groups are generally not specified, but may be present nonetheless.

The term “immunoglobulin” refers to a protein consisting of one or more polypeptide(s) substantially encoded by immunoglobulin genes. The recognized immunoglobulin genes include the different constant region genes as well as the myriad immunoglobulin variable region genes. Immunoglobulins may exist in a variety of formats, including, for example, Fv fragments, Fab fragments, and F(ab)₂ fragments as well as single chain fragments (scFv) or diabodies (e.g. Huston, J. S., et al., Proc. Natl. Acad. Sci. USA 85 (1988) 5879-5883; Bird, R. E., et al., Science 242 (1988) 423-426; in general, Hood et al., Immunology, Benjamin N.Y., 2nd edition (1984); and Hunkapiller, T. and Hood, L., Nature 323 (1986) 15-16).

A full length immunoglobulin in general comprises two so called light chain polypeptides (light chain) and two so called heavy chain polypeptides (heavy chain). Each of the heavy and light chain polypeptides contains a variable domain (variable region) (generally the amino terminal portion of the polypeptide chain) comprising binding regions that are able to interact with an antigen. Each of the heavy and light chain polypeptides comprises a constant region (generally the carboxyl terminal portion). The constant region of the heavy chain mediates the binding of the antibody i) to cells bearing a Fc gamma receptor (FcγR), such as phagocytic cells, or ii) to cells bearing the neonatal Fc receptor (FcRn) also known as Brambell receptor. It also mediates the binding to some factors including factors of the classical complement system such as component (Clq).

The variable domain of an immunoglobulin's light or heavy chain in turn comprises different segments, i.e. four framework regions (FR) and three hypervariable regions (CDR).

In one embodiment the biologically active polypeptide is an immunoglobulin fragment.

The term “immunoglobulin fragments” denotes a portion of a full length immunoglobulin, in one embodiment the variable domains thereof or at least the antigen binding portion thereof. An immunoglobulin fragment retains the binding characteristics of the parental full length immunoglobulin with respect to its antigen(s). Examples of immunoglobulin fragments are e.g. single-chain antibody molecules (scFv), Fab, F(ab)₂ fragments, and the like as long as they retain the binding characteristics of the parental full length immunoglobulin.

In one embodiment the polypeptide is a toxin. In one embodiment the polypeptide is an immunoglobulin-toxin conjugate. In one embodiment the polypeptide is an immunoglobulin fragment-toxin conjugate. In one embodiment the polypeptide is a hormone. In one embodiment the polypeptide is a cytokine.

The aim of the production scientist is to increase the yield in recombinant polypeptide production.

The production yield increases achievable using improved media compositions and cultivation techniques will be at an end sometime in the future. Therefore metabolic engineering of production cell lines and strains will become more important.

Diverse methods for the targeted inactivation of genes in prokaryotic organisms are known. One example is the Red/ET recombination method. In this method the target nucleic acid is modified (i.e. replaced and deleted) by homologous recombination mediated by bacteriophage derived polypeptides.

The terms “respiratory chain” or “respiratory chain enzyme” (being an enzyme which is involved in the respiratory chain) is known to a person skilled in the art and is described e.g. in Berg, J M et al. (Biochemistry, 5^{th} Edition, 2002). Exemplary respiratory chain enzymes are e.g. NADH dehydrogenases, Sox type oxidases (like SoxM type oxidase or SoxB type oxidase), cytochrome bd type oxidase or cytochrome bo type oxidase.

The NADH dehydrogenase II (encoded by the ndh-gene) is involved in the transfer of electrons from NADH into the respiratory chain. The transfer is coupled to a proton gradient via the quinone pool and uses the bo-type and the bd-type oxidase in parallel for the final electron transfer to oxygen.

The NADH dehydrogenase II has a “sister”-enzyme the NADH dehydrogenase I. The activities of NADH dehydrogenase I and II depend to a varying extent on the proton gradient resulting indifferent H⁺/e⁻ ratios.

It has been found that by the inactivation of the ndh-gene the (specific) productivity of an E. coli cell can be increased whereby surprisingly the oxygen uptake rate and the growth rate remain comparable to those of the parent E. coli cell that is isogenic with the ndh-deficient E. coli cell except for the ndh-gene.

It has been found that the inactivation of the ndh-gene in an E. coli cell (of the genotype 1) resulting in an ndh-deficient (NADH dehydrogenase II-deficient) modified E. coli cell (of genotype 1 Δndh) has a profound impact on the (specific) productivity of the E. coli cell, which is increased compared to the parent E. coli cell. At the same time the oxygen uptake rate and the growth rate is comparable between the modified E. coli cell and the parent E. coli cell.

The term “comparable” denotes that two values are within 50% of each other. In one embodiment the values are within 30% of each other. In one embodiment the values are within 10% of each other. For example, two values are within 50% of each other and are, thus, comparable when the second value does not exceed the first value by more than 50%, i.e. is not more than 150% of the first value, and when the second value is not less than 50% of the first value, i.e. comparable denotes that the second value is between 50% and 150% of the first value.

The deletion/inactivation of the ndh-gene results in an ndh-deficient cell (genotype Δndh).

In FIG. 1 it can be seen that the parent E. coli and the modified E. coli grow to comparable cell densities in comparable time. Thus, the deletion/inactivation of the ndh-gene has no negative influence on the growth characteristics of the modified E. coli (compared to the parent E. coli).

In FIG. 2 it can be seen that the parent E. coli and the modified E. coli have a comparable oxygen uptake rate during the cultivation. Thus, the deletion/inactivation of the ndh-gene has no influence on the oxygen demand of the modified E. coli (compared to the parent E. coli).

In FIG. 3 it can be seen that the modified E. coli has a higher (specific) production rate resulting in a higher product concentration compared to the parent E. coli. Thus, the deletion/inactivation of the ndh-gene has a positive effect on the production rate of the modified E. coli.

### DESCRIPTION OF THE FIGURES

FIG. 1 Growth characteristics of parent E. coli (filled diamonds, genotype 1) and modified E. coli (filled squares, genotype 1 Δndh).

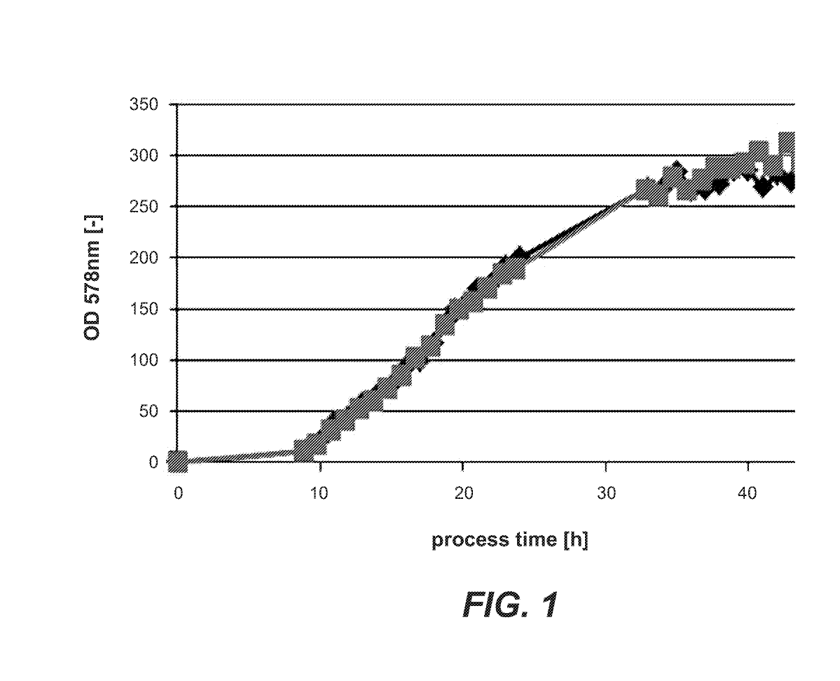

FIG. 2 Oxygen uptake rate parent E. coli (lower curve, genotype 1) and modified E. coli (upper curve, genotype 1 Δndh) determined at 20 hours cultivation time.

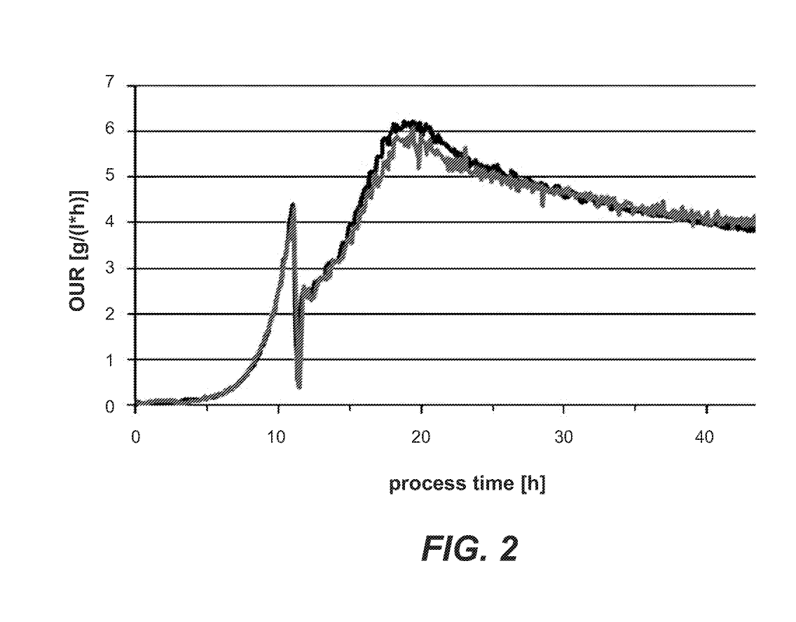

FIG. 3 Production rate of parent E. coli (filled diamonds, genotype 1) and modified E. coli (filled squares, genotype 1 Δndh).

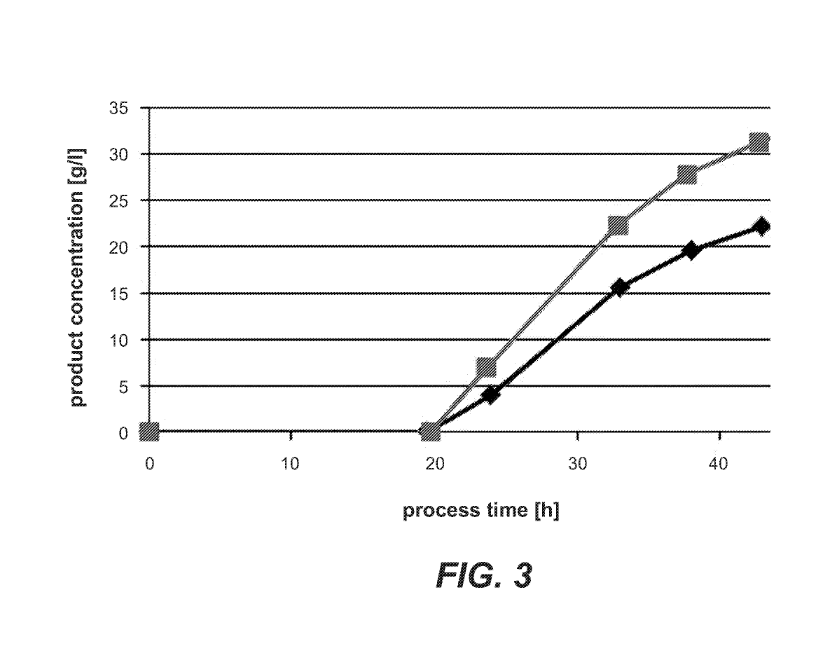

The following examples and figures are provided to aid the understanding of the present invention, the true scope of which is set forth in the appended claims. It is understood that modifications can be made in the procedures set forth without departing from the spirit of the invention.

### EXAMPLE 1

### Making and Description of the E. coli Expression Plasmids

The shortened tetranectin-apolipoprotein A-I fusion protein was prepared by recombinant means. The expressed fusion protein has in N- to C-terminal direction the amino acid sequence of SEQ ID NO: 01:

PIVNAKKDVVNTKMFEELKSRLDTLAQEVALLKEQQALQTVDEPPQSP
WDRVKDLATVYVDVLKDSGRDYVSQFEGSALGKQLNLKLLDNWDSVTS
TFSKLREQLGPVTQEFWDNLEKETEGLRQEMSKDLEEVKAKVQPYLDD
FQKKWQEEMELYRQKVEPLRAELQEGARQKLHELQEKLSPLGEEMRDR
ARAHVDALRTHLAPYSDELRQRLAARLEALKENGGARLAEYHAKATEH
LSTLSEKAKPALEDLRQGLLPVLESFKVSFLSALEEYTKKLNTQ.

The encoding fusion gene is assembled with known recombinant methods and techniques by connection of appropriate nucleic acid segments. Nucleic acid sequences made by chemical synthesis are verified by DNA sequencing. The expression plasmid for the production of the fusion protein of SEQ ID NO: 01 can be prepared as follows:

Plasmid 1 (1-pBRori-URA3-LACI-SAC) is an expression plasmid for the expression of core-streptavidin in E. coli. It was generated by ligation of the 3142 by long EcoRI/CelII-vector fragment derived from plasmid 2 (2-pBRori-URA3-LACI-T-repeat; reported in EP-B 1 422 237) with a 435 by long core-streptavidin encoding EcoRI/CelII-fragment.

The core-streptavidin E. coli expression plasmid comprises the following elements:
the origin of replication from the vector pBR322 for replication in E. coli (corresponding to by position 2517-3160 according to Sutcliffe, G., et al., Quant. Biol. 43 (1979) 77-90),
the URA3 gene of Saccharomyces cerevisiae coding for orotidine 5′-phosphate decarboxylase (Rose, M., et al., Gene 29 (1984) 113-124) which allows plasmid selection by complementation of E. coli pyrF mutant strains (uracil auxotrophy),
the core-streptavidin expression cassette comprising
   the T5 hybrid promoter (T5-PN25/03/04 hybrid promoter according to Bujard, H., et al., Methods. Enzymol. 155 (1987) 416-433 and Stueber, D., et al., Immunol. Methods IV (1990) 121-152) including a synthetic ribosomal binding site according to Stueber, D., et al. (see before),
   the core-streptavidin gene,
   two bacteriophage-derived transcription terminators, the λ-T0 terminator (Schwarz, E., et al., Nature 272 (1978) 410-414) and the fd-terminator (Beck, E. and Zink, B., Gene 1-3 (1981) 35-58),
the lacI repressor gene from E. coli (Farabaugh, P. J., Nature 274 (1978) 765-769).

The final expression plasmid for the expression of the shortened tetranectin-apolipoprotein A-I fusion protein can be prepared by excising the core-streptavidin structural gene from plasmid 1 using the singular flanking EcoRI and CelII restriction endonuclease cleavage site and inserting the EcoRII/CelII restriction site flanked nucleic acid encoding the fusion protein into the 3142 by long EcoRI/CelII-1 plasmid fragment.

### EXAMPLE 2

### Comparison of the Parental Strain to the Ndh-Deletion Mutant Strain in Expressing a Recombinant Polypeptide

To evaluate the effect of the chromosomal ndh gene deletion on the performance of an E. coli strain expressing a recombinant protein in high cell density and high yield fermentation compared the parental strain was compared with the modified strain within the same process and explored growth and product formation.

The E. coli K12 parental strain (genotype: thi-1, ΔompT, ΔpyrF, acnA, aceA, icd) and the modified strain (genotype: thi-1, ΔompT, ΔpyrF, Δndh, acnA, aceA, icd) were transformed by electroporation with the final expression plasmid as described in Example 1 to express a TN-ApoA1 fusion polypeptide. Herein, the acnA gene encoded polypeptide comprises a S68G mutation, the aceA gene encoded polypeptide comprises a S522G mutation and the icd gene encoded polypeptide comprises a D398E and a D410E mutation. In addition the parental and the modified strain lack the following e14 prophage genes: ymfD, ymfE, lit, intE, xisE, ymfI, ymfJ, cohE, croE, ymfL, ymfM, owe, ymfR, bee, jayE, ymfQ, stfP, tfaP, tfaE, stfE, pinE, mcrA. The transformed E. coli cells were first grown at 37° C. on agar plates. A colony picked from this plate was transferred to a 3 mL roller culture and grown at 37° C. to an optical density of 1-2 (measured at 578 nm). Then 1000 μL culture where mixed with 1000 μL sterile 86%-glycerol and immediately frozen at −80° C. for long time storage. The correct product expression of this clone was first verified in small scale shake flask experiments and analyzed with SDS-Page prior to the transfer to the 10 L fermenter.

Pre Cultivation in Chemically Defined Medium (CDM):

For Pre-Fermentation a Chemical Defined Medium has been Used:

NH4Cl 1.0 g/L, K2HPO4*3H2O 18.3 g/L, citrate 1.6 g/L, Glycine 0.78 g/L, L-Alanine 0.29 g/L, L-Arginine 0.41 g/L, L-Asparagine*H2O 0.37 g/L, L-Aspartate 0.05 g/L, L-Cysteine*HCl*H2O 0.05 g/L, L-Histidine 0.05 g/L, L-Isoleucine 0.31 g/L, L-Leucine 0.38 g/L, L-Lysine*HCl 0.40 g/L, L-Methionine 0.27 g/L, L-Phenylalanine 0.43 g/L, L-Proline 0.36 g/L, L-Serine 0.15 g/L, L-Threonine 0.40 g/L, L-Tryptophan 0.07 g/L, L-Valine 0.33 g/L, L-Tyrosine 0.51 g/L, L-Glutamine 0.12 g/L, Na-L-Glutamate*H2O 0.82 g/L, Glucose*H2O 6.0 g/L, trace elements solution 0.5 ml/L, MgSO4*7H2O 0.86 g/L, Thiamin*HCl 17.5 mg/L. The trace elements solution contains FeSO4*7H2O 10.0 g/L, ZnSO4*7H2O 2.25 g/L, MnSO4*H₂O 2.13 g/L, H3BO3 0.50 g/L, (NH4)6Mo7O24*4H2O 0.3 g/L, CoCl2*6H2O 0.42 g/L, CuSO4*5H2O 1.0 g/L dissolved in 0.5M HCl.

For pre-fermentation 300 ml of CDM-medium in a 1000 ml Erlenmeyer-flask with four baffles was inoculated with 0.9 ml out of a primary seed bank ampoule. The cultivation was performed on a rotary shaker for 8 hours at 32° C. and 170 rpm.

Fermentation Process (AP30#021 and AP50#001):

| cultivation | genotype | batch | comment |
|---|---|---|---|
| 1 | thiE, | AP30#021 | reference |
| | ΔpyrF, | | fermentation |
| | acnA, | | |
| | aceA, icd | | |
| 2 | thiE, | AP50#001 | fermentation with |
| | ΔpyrF, | | ndh-mutant |
| | Δndh, | | comparable to |
| | acnA, | | AP30#021 |
| | aceA, icd | | |

For fermentation in a 10 L Biostat C, DCU3 fermenter (Sartorius, Melsungen, Germany) the following batch medium was used: KH₂PO₄ 1.58 g/L, (NH₄)₂HPO₄ 7.47 g/L, K2HPO4*3H2O 13.32 g/L, citrate 2.07 g/L, L-Methionine 1.22 g/L, NaHCO₃ 0.82 g/L, trace elements solution 7.3 ml/L, MgSO₄*7 H₂O 0.99 g/L, Thiamine*HCl 20.9 mg/L, glucose*H₂O 29.3 g/L, Biotin 0.2 mg/L, 1.2 ml/L Synperonic 10% anti foam agent. The trace elements solution contains FeSO4*7H2O 10 g/L, ZnSO4*7H2O 2.25 g/L, MnSO4*H₂O 2.13 g/L, CuSO4*5H2O 1.0 g/L, CoCl2*6H2O 0.42 g/L, (NH4)6Mo7O24*4H2O 0.3 g/L, H3BO3 0.50 g/L solubilized in 0.5M HCl solution.

The feed 1 solution contained 700 g/L glucose*H2O, 7.4 g/L MgSO₄*7 H₂O and 0.1 g/L FeSO4*7H2O. Feed 2 comprises KH₂PO₄ 52.7 g/L, K2HPO4*3H2O 139.9 g/L and (NH4)2HPO4 66.0 g/L. All components were dissolved in deionized water. The alkaline solution for pH regulation was an aqueous 12.5% (w/v) NH₃ solution supplemented with 11.25 g/L L-Methionine.

Starting with 4.2 L sterile batch medium the batch fermentation was performed at 31° C., pH 6.9±0.2, 800 mbar back pressure and an initial aeration rate of 10 L/min. The relative value of dissolved oxygen (pO2) was kept at 50% throughout the fermentation by increasing the stirrer speed up to 1500 rpm. After the initially supplemented glucose was depleted, indicated by a steep increase in dissolved oxygen values, the temperature was shifted to 25° C. and 15 minutes later the fermentation entered the fed-batch mode with the start of both feeds (60 and 14 g/h respectively). The rate of feed 2 is kept constant, while the rate of feed 1 is increased stepwise with a predefined feeding profile from 60 to finally 160 g/h within 7 hours. When carbon dioxide off gas concentration leveled above 2% the aeration rate was constantly increased from 10 to 20 L/min within 5 hours. The expression of recombinant tetranectin-apolipoprotein A-I fusion protein was induced by the addition of 2.4 g IPTG at an optical density of approx. 150.

At the end of fermentation the within the cytoplasm soluble expressed tetranectin-apolipoprotein A-I is transferred to insoluble protein aggregates, the so called inclusion bodies, with a heat step where the whole culture broth in the fermenter is heated to 50° C. for 1 hour before harvest (see e.g. EP-B 1 486 571). Thereafter, the content of the fermenter was centrifuged with a flow-through centrifuge (13,000 rpm, 13 L/h) and the harvested biomass was stored at −20° C. until further processing. The synthesized tetranectin-apolipoprotein A-I fusion proteins were found exclusively in the insoluble cell debris fraction in the form of insoluble protein aggregates, so-called inclusion bodies (Ms).

Analysis of Product Formation:

Samples drawn from the fermenter, one prior to induction and the others at dedicated time points after induction of protein expression are analyzed with SDS-Polyacrylamide gel electrophoresis. From every sample the same amount of cells (OD_{Target}=5) are suspended in 5 mL PBS buffer and disrupted via sonication on ice. Then 100 μL of each suspension are centrifuged (15,000 rpm, 5 minutes) and each supernatant is withdrawn and transferred to a separate vial. This is to discriminate between soluble and insoluble expressed target protein. To each supernatant (=soluble) fraction 300 μL and to each pellet (=insoluble) fraction 400 μL of SDS sample buffer (Laemmli, U.K., Nature 227 (1970) 680-685) are added. Samples are heated for 15 minutes at 95° C. under intense mixing to solubilize and reduce all proteins in the samples. After cooling to room temperature 5 μL of each sample are transferred to a 4-20% TGX Criterion Stain Free polyacrylamide gel (Bio-Rad). Additionally 5 μL molecular weight standard (Precision Plus Protein Standard, Bio-Rad) and 3 amounts (0.3 0.6 μL and 0.9 μL) quantification standard with known product protein concentration (0.1 μg/μL) are positioned on the gel.

The electrophoresis was run for 60 Minutes at 200 V and thereafter the gel was transferred the GelDOC EZ Imager (Bio-Rad) and processed for 5 minutes with UV radiation. Gel images were analyzed using Image Lab analysis software (Bio-Rad). With the three standards a linear regression curve was calculated with a coefficient of >0.99 and thereof the concentrations of target protein in the original sample was calculated.

Results:

The above mentioned fermentation process was used to express a shortened tetranectin-apolipoprotein A-I fusion protein in the parental strain and in the modified strain representing the ndh deletion mutant. Despite the optical density of the pre-culture of the modified strain was lower the growth of both strains was very comparable. After 47 hours of cultivation and the consecutive heat step optical densities of 285 and 245 were obtained.

The modification of the ndh expression should result in a decreased oxygen uptake rate (OUR) as described by Calhoun et al. (J. Bacteriol. 175 (1993) 3020-3025). Surprisingly the modified strain had an almost comparable OUR as the parent strain in this experiment under the same cultivation conditions. In the first period of the fed-phase of fermentation the OUR of the modified strain was even higher when compared to the parental strain.

Product formation was induced by the addition of 2.4 g IPTG at an optical density of approx. 150 in both attempts.

Despite both strains were cultivated on the same chemically defined medium and under the same conditions the product formation rate of the parent strain was significant lower and therefore the final yield reached only 27.5 g/L. In comparison to that the modified strain had a significantly higher product formation rate. This is not expected when looking only on the data of growth and OUR in direct comparison with the parental strain. The same amount of target protein was produced by the ndh-deficient modified strain after only 38 hours of cultivation (27.8 g/L) and the fermentation could be terminated 10 hours earlier than when using the parent strain to produce the polypeptide TN-ApoA1. In addition the cultivation with the ndh-deficient modified strain yielded in 8.4% more (29.8 g/L) fusion protein at the end of fermentation and after the heat step. The parental E. coli strain has significant deficits in direct comparison with the modified strain.

Summary:

Despite both strains were showing the same growth in fermentation on chemical defined medium the ndh-deficient modified strain had an unexpected increase in oxygen uptake rate during the fed-batch phase of the process and a significantly higher product formation rate. Therefore the final product yield could be increased. Because the only difference in both experiments was the modification in the ndh gene locus of the ndh-deficient modified strain this effect can directly be correlated to that. Therefore it is useful to delete ndh in highly productive E. coli strains not to reduce OUR but to increase productivity.

1. A method for the recombinant production of a polypeptide in E. coli comprising the following steps:
cultivating an NADH dehydrogenase II-deficient E. coli expressing the polypeptide, and
recovering the polypeptide from the cell or the cultivation medium,
wherein the polypeptide is not a respiratory chain pathway enzyme or a polypeptide encoded by an antibiotic resistance inducing gene.

2. The method according to claim 1, characterized in that the NADH dehydrogenase II-deficient E. coli has a comparable oxygen uptake rate as an E. coli with the same genotype except that it has a functional NADH dehydrogenase II.

3. The method according to any one of claims 1 to 2, characterized in that the NADH dehydrogenase II-deficient E. coli has a comparable growth rate as an E. coli with the same genotype except that it has a functional NADH dehydrogenase II.

4. The method according to any one of claims 1 to 3, characterized in that the NADH dehydrogenase II-deficient E. coli has a higher production rate as an E. coli with the same genotype except that it has a functional NADH dehydrogenase II.

5. The method according to any one of claims 1 to 4, characterized in that the NADH dehydrogenase II-deficient E. coli is an E. coli K12.

6. The method according to claim any one of claims 1 to 5, characterized in that the NADH dehydrogenase II-deficient E. coli has the genotype thi-1, Δndh, ΔpyrF.

7. The method according to any one of claims 1 to 6, characterized in that the NADH dehydrogenase II-deficient E. coli is further deficient in the bd-type oxidase.

8. An E. coli K12 that has the genotype thi-1, Δndh, ΔpyrF.

9. Use of an NADH dehydrogenase II-deficient E. coli in the production of a recombinant polypeptide, wherein the polypeptide is not a respiratory chain pathway enzyme or a polypeptide encoded by an antibiotic resistance inducing gene.

10. The use according to claim 9, characterized in that the NADH dehydrogenase II-deficient E. coli has the genotype thi-1, Δndh, ΔpyrF.

11. The use according to any one of claims 9 to 10, characterized in that the NADH dehydrogenase II-deficient E. coli is further deficient in the bd-type oxidase.


SEQUENCE LISTING (1 sequence)

SEQ ID NO: 1 - protein, 284 aa, Artificial Sequence
  tetranectin-apolipoprotein A-I fusion protein
PIVNAKKDVVNTKMFEELKSRLDTLAQEVALLKEQQALQTVDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ